(19) United States
(12) Patent Application Publication
(10) Pub. No.: US 2012/0129152 A1
(43) Pub. Date: May 24, 2012

(54) Diagnostic method for the prediction of the development of and control over the effectiveness of treatment of cardiovascular illnesses

(76) Inventors: Artur Martynov, Kharkov (UA); Boris S. Farber, Brooklyn, NY (US); Sonya Sophya Farber, New York, NY (US)

(21) Appl. No.: 12/931,460

(22) Filed: Feb. 1, 2011

Related U.S. Application Data
(63) Continuation of application No. PCT/RU2010/000695, filed on Nov. 22, 2010.

(51) Int. Cl. C12Q 1/70

(52) U.S. Cl. 435/5

(57) ABSTRACT

A diagnostic method for the prediction of the development and control of the effectiveness of the treatment of cardiovascular diseases, in which patient tissue samples are taken, microassay are prepared, specific antiviral immunoglobulins are processed, the number of cells infected by two or more viruses before the beginning of treatment are determined, and the dynamic of the change in the number of infected cells and their interrelationships are established: when the number of cells infected by cytomegalovirus and any other viruses decreases by more than 50±10% in patients without symptoms of cardiovascular pathology, a diagnostic conclusion is high danger of the development of atherosclerosis; if the number of cells infected by cytomegalovirus and any other viruses exceeds 50±10% in patients with demonstrated clinical signs of cardiovascular system pathology, a diagnostic conclusion is drawn of the danger of the development of complications such as arrhythmia, thrombolytic embolism.

### TECHNICAL FIELD

This invention is related to medicine—specifically, to cardiology—and is intended to predict the development and to control the effectiveness of treatment of cardiovascular diseases in humans. It will permit the prediction of the possibility that complications of atherosclerosis will develop and improve the effectiveness of its treatment.

Previous Level of Technology

Cardiovascular diseases (including atherosclerosis and its complications) are a major cause of death for people in developed countries [¹]. For Ukraine, this problem is even more pressing: according to the WHO, in Ukraine in 2005, cardiovascular disease (CVD) was the reason for approximately 60% of all deaths, which significantly exceeds the analogous indicator in the developed countries of Europe (38%) [²]. Therefore, any new information on the mechanisms of development for atherosclerosis, as well as the development of new, more effective methods for its treatment, are of momentous social importance.

In accordance with the modern concept of medical science, atherosclerosis is a chronic inflammatory disease of the walls of the large arteries [³, ⁴, ⁵]. In atherosclerosis, lipids, fibroid elements, and calcium salts collect in the arteries, which leads to a decrease in the clearance of the arteries and in their elasticity. In principle, this process begins almost at birth and accompanies the inevitable process of the body's aging [⁶, ⁷, ⁸]. The development of atherosclerosis begins with damage to the endothelium of blood vessels and the disruption of its functions. The damaged wall of the blood vessel, in addition to a local compensatory reaction, initiates a powerful systemic response in the form of a cascade of molecular reactions and cell processes such as a expression of molecular adhesion, hemotaxis factors, inflammatory cytokines, and growth factors, involvement of leukocytes, expression by the bone marrow of progenitor cells and their pickup by the vessel wall to eliminate the damage, and so on [⁹]. Normally, this process leads to the restoration of the function of the endothelium and homeostasis; however, in many cases, when an unfortunate combination of risk factors comes together, the process may become pathological, leading to a destructive systemic inflammatory reaction.

A few risk factors are known, both genetically conditioned and external in nature, that cause damage to the endothelium and activate atherogenesis: an increased cholesterol level (in part, the low-density lipoproteins, LDLs), an increased level of homocysteine, arterial hypertension, diabetes, smoking, genetic disposition, and age (aging). The process of atherogenesis is a long and complex one; several detailed overview articles are dedicated to this topic [¹⁰]. The following stages may be determined in the development of atherosclerosis.

Stage I: Fatty streaks: aggregation of LDL particles in the subendothelial layer (intima) of the blood vessel. LDL is subject to acidification (the process is stimulated by free radicals), and in that form, the lipoprotein penetrates to the intima more actively. LDL plays a key role in this process: the higher its level, the faster atherogenesis takes place. Further, with the active participation of inflammation mediators (interleukine-1, cachectin α, growth factures, adhesion molecules), an active migration of monocytes and T-lymphocytes to the location of the microscopic damage to the endothelium takes place. They penetrate to the intima, the monocytes turn into macrophages, and the macrophages pick up the acidized LDL. Thus foam cells are formed. The sections of the massive aggregation of foam cells in this stage can be visualized as fatty streaks.

Stage II: Formation of a necrotic core. With time, the foam cells die off, and their lipoprotein-containing remains collect in the intima and as a result form a necrotic core of atherosclerotic disease. Sometimes smooth muscle cells (SMCs) embed themselves in the fatty streaks, migrating from the medial layer of the blood vessel.

Stage III: Intracellular aggregation of lipoprotein. The acidified LDL continues to aggregate in the intima; the atherosclerotic plaque grows in size.

Stage IV: Formation of a lipid core. The buildup of SMCs continues; in the process of the aggregation of lipoprotein in the intima, calcification becomes involved, taking place first inside the SMCs, and after their death, between the muscle wall of the blood vessel and the external part of the atherosclerotic plaque.

Stage V: Formation of an atheroma. Between the fat deposit and the intima, a protective layer forms, consisting primarily of fibrin and collagen filaments. This encapsulation of the fat deposit is called an atheroma. For a certain amount of time, the atheroma grows inside the vessel wall, causing a compensatory expansion of the vessel, but after it reaches a critical size, it begins to encroach on the vessel's opening, decreasing its diameter and affecting blood flow; stenosis is developed. It should be noted that before this stage (and later, in many cases) the process of the development of atherosclerosis goes on asymptomatically and lacks almost all clinical manifestation; only stenosis of more than 77% is considered to be the limit in cardiology after which a clinically expressed disease begins.

Stage VI: Rupture of the atheroma and thrombosis. When the integrity of the fibral capsule covering the atheroma is breached, thrombocytes and tissue factors are set free, leading to a cascade of biochemical blood clotting reactions. A clot is thus formed. Whereas the duration of the previous stages of atherosclerotic development may have been from several weeks to decades, the creation of the clot may occur within scant minutes, leading to an embolism and often causing catastrophic consequences in the form of a heart attack or stroke. According to clinical study data, only about 14% of clinically expressed phenomena occur at a compression of the blood vessel openings of 77% and higher; the majority of life-threatening and deadly events occur as the result of the abruption of the plaque and the following thrombosis [¹¹].

Infectious components in the etiology of atherosclerosis. Notwithstanding the fact that the risk factors of atherosclerosis are well-known these days, even all of them together cannot explain half of the clinical cases of atherosclerosis [¹²]. Thus infectious diseases are being entered in the list of risk factors with increasing certainty. The infectious etiology was confirmed for many chronic illnesses, such as stomach ulcers (Heliobacter pylori), cervical cancer (various papilloma viruses) and liver cancer (the hepatitis B and C viruses). Increasing amounts of data indicate the participation of infection in the etiology of diabetes mellitus, Alzheimer's disease, various neurological diseases, and cardiovascular disease [¹³]. The hypothesis on the infectious nature of atherosclerosis first appeared in the 19th century in the works of R. Virchow [¹⁴], and later, in 1889, in the works of Gilbert and Lion. The association of atherosclerosis and viral infection was empirically grounded in the 1970s in the works of C. G. Fabricant et al, in which atherosclerosis-like changes in the blood vessels developed in chicks infected with the Marek's disease virus, an avian variant of the herpes virus [¹⁵]. Many viruses have been proposed in the role ofpossible atherogenesis pathogens: cytomegalovirus (CMV) [¹⁶, ¹⁷, ¹⁸, ¹⁹, ²⁰, ²¹, ²²], herpes simplex (HSV) [²³, ²⁴] and Epstein-Barr [²⁵, ²⁶] and many others. The majority of experimental studies of the association of infections and atherosclerosis have been dedicated to CH pneumoniae and cytomegalovirus. Both of these microorganisms are highly likely to be found in vessels with atherosclerosis and atherosclerotic plaques, and being seropositive for them correlates well with atherosclerosis and severe courses of cardiovascular disease [²⁷, 12, ²⁸].

Regardless of the fact that the association of two infectious agents—Ch. pneumoniae and cytomegalovirus—with cardiovascular diseases can be considered proven, this does not indicate a cause and effect connection. Moreover, Koch's classic postulates cannot be fulfilled in the case of a multi-factor disease like atherosclerosis [1,31]. Most likely, the infections are neither necessary nor a sufficient reason for the development of atherosclerosis, but they are a risk factor that increases the likelihood of the development of the pathology. In order to prove that infections participate in atherosclerosis, in addition to the histopathological data, a precise concept of the mechanism(s) of the pathogenesis, experimentally confirmed in vitro and in vivo, and as a result, of the methods of treatment, the effectiveness of which has been proven in experiment and in clinical conditions, are needed. Many mechanisms of pathogenic activity of infectious agents on the cardiovascular system are known:²⁹]:
an increase in the proliferation of HM cells and in their migration (human cytomegalovirus)
protection of the cells of the endothelium from apoptosis, which leads to their excess aggregation and an increase in the size of the atherosclerotic plaque (cytomegalovirus, Ch. pneumoniae)
increased speed of lipid aggregation (cytomegalovirus, Ch. pneumoniae)
an increase in the pro-coagulation activity of the endothelial cells (cytomegalovirus, HSV)
an increase in the expression of cytokines, chemokines, and adhesion molecules, an appearance of severe phase proteins (C-reactive protein, serum amyloid C, etc.), which leads to a vicious cycle of hyperergic inflammatory reaction, and, as a result, to damage to the endothelium (nearly all pathogenic microorganisms are suspected)
an increase in the level of reactive forms of oxygen (oxygen ions, free radicals, peroxides) caused by increased acidization of LDL (cytomegalovirus)
autoimmune reactions; certain proteins created by pathogens that are homologous to proteins found in the human body, which may be the reason for the arousal of an autoimmune reaction, a high level of homologic proteins are seen for the Heat Shock Protein (HSP) of various bacteria and humans (HSP60)

A good deal of data obtained in animal experiments confirms the results of the histopathology and in vitro research. According to the data of C. G. Fabricant et al [15], the Marek's disease virus caused atherosclerosis in chicks, and the immunization of healthy animals prevented the development of the pathology regardless of the amount of cholesterol in the feed taken. They also studied the effect of immune stimulation therapy on the development of atherosclerosis in rabbits experimentally infected with HSV: a significant slowing in the atherosclerosis development process was noted [16]. A significant amount of data obtained as a result of histopathological analysis as well as in vitro and in vivo in the experiment confirms the hypothesis of the infectious component in the etiology of atherosclerosis.

The next stage in the development of the concept of infectious components in the etiology of atherosclerosis was clinical trials with the use of antibiotics. Since the majority of the experimental data bore witness to the involvement of Ch. pneumoniae in atherosclerosis, the majority of the experiments were directed at eliminating it. The most effective medicines against Ch. pneumoniae are believed to be antibiotics from the macrolide group, including azithromycin, due to their ability to penetrate inside the cell and act on intracellular parasites [³⁰, ³¹]. From 1997 through 2005, many clinical trials were conducted using azithromycin (or other macrolides: roxithromycin and clarithromycin) as one of the agents for the compound treatment of cardiovascular disease (ischemic heart disease, old myocardial infarction, and unstable stenocardia) [³², ³³, ³⁴, ³⁵]. Out of the more than 100 studies, eleven were promising, randomized, and placebo-controlled. In them, the effect of antibiotic therapy on the frequency of appearance of myocardial infarction and stroke, their repeated development, and the overall mortality rate as a result of cardiovascular disease were evaluated [33]. A comparison of the results of the studies held is a difficult task due to the variations in the criteria for the inclusion of patients, the design of the studies themselves, and the duration of observation after treatment. Even so, a conclusion may be drawn from the results of an analysis of the studies: notwithstanding the fact that after the first pilot studies, encouraging results were achieved, more large-scale clinical studies did not demonstrate any advantage whatsoever to antibiotic therapy in the treatment of cardiovascular disease.

Atherosclerosis is a widespread chronic disease with a multi-factor and not fully explained etiology and a long and secret pathogenesis. The evolution of the understanding of the reasons for and mechanisms of this disease's appearance by modem science is developing quickly, although it is insufficient to the demands of the times. In the 1990s, it was hoped that cardiovascular diseases could be eliminated by the end of the century through control over cholesterol and arterial pressure; ten years later, it became clear that this optimistic prognosis needed to be reexamined [10]. Thus a change in paradigms occurred, as the result of which atherosclerosis ceased to be a simple disruption to the metabolism by cholesterol and was recognized as an inflammatory disease [³⁶, 10]. Traditional and prospective methods for the treatment of atherosclerosis through drugs are directed toward various links in the pathogenetic chain, such as lipid exchange (statins, fibrates, cholesterol absorption inhibitors), blockade of renin-angiotensin systems (angiotensin converting enzyme inhibitors [ACEI]), blockade of 3-adrenoreceptors and excess calcium accumulation (calcium ion antagonists), oxidative stress (tocopherol analogues: vitamin E, probucol, AGI-1067), excess proliferation of HM cells (HM cell growth inhibitor), aggregation of thrombocytes (thrombocyte activation inhibitors), inflammatory processes in the blood vessels (acetosalicylic acid, AGI-1067), and thrombosis (anti-thrombotic drugs, heparin, thrombin antagonists). The most effective are the drugs with a pleiotropic effect: that is, those that act simultaneously on various mechanisms of pathogenesis such as statins (lipid exchange, antiatherosclerotic, anti-inflammatory, and anti-thrombolytic action) and the new class of therapeutic agents, vascular protectants, which have antioxidant and anti-inflammatory properties and also decrease post-angioplasty restenosis [³⁷].

In addition, even in a complex approach and with the use of new pleiotropic drugs, the treatment of atherosclerosis remains, as before, pathogenetic. It is possible that the next big moment in the evolution of atherosclerosis and cardiovascular disease treatment will be the reexamination of the concept of the therapy and a transition from pathogenetic to etiotropic and pathogenetic treatment, as well as to preventative treatment (in the early stages before the manifestation of clinical symptoms), with the goal of preventing the development of severe forms of atherosclerosis. One of the types of this therapy could be treatment of infections that are risk factors for the development of atherosclerosis (Ch. pneumoniae, HSV, and CMV). Interferon and its inducers have been effective in the treatment of viral myocardias of various etiologies caused by entero- and adenoviruses, as well as by herpes viruses [³⁸]. These in vitro studies are promising; however, in order to implement atherosclerosis therapy with the use of interferons, large-scale clinical trials are needed, as well as determination of groups of patients and stages of illness for which this therapy would be most effective.

The results of many studies indicate that infectious diseases heavily influence the etiology and pathogenesis of atherosclerosis, but data on the supposed causes of the diseases are contradictory. Considering that atherosclerosis is a multi-factor disease, Koch's postulates are not applicable in this case; most likely a single infection will not be discovered that is a trigger for atherosclerosis. The most likely risk factor is the “infection load” factor. Data from many studies indicate a direct correlation between the number of infections discovered in a single patient and the level of severity of cardiovascular diseases, as well as mortality due to cardiovascular diseases.

Clinical studies conducted using antibiotics did not facilitate a decrease in the likelihood of infarction and mortality due to cardiovascular diseases. There may be many reasons for this, but most likely, it is that the activity of the antibiotics was directed toward one infection only, while the main role in the development of atherosclerosis is played by herpesvirus infections. Use of the most accessible tablet form of the herpes drug Valacyclovir and its injectible counterpart in complex therapy for the diseases caused by atherosclerosis may significantly improve the prognosis of the course of the disease, increase remission periods, and prevent relapses of heart attacks and strokes.

A method of discovering circulating proteins that are differentially expressed in atherosclerosis is known. The circulation levels of these proteins, especially sets of proteins, may allow the discovery of patients with severe myocardial infarction and their differentiation from those with stable stenocardia. The sets of proteins discovered may permit the prediction of cardiovascular complications and the prediction and control of the effectiveness of the therapy, the stage of the disease, and so on. For example, these markers are beneficial in combination with clinical data for the development of specific pharmacotherapy schemes [³⁹]. The method's shortcoming is the accent on the change of pathogenetic markers. Also, the method does not take into account the effect of etiological viral factors on the progression of the disease and the effectiveness of its treatment, which does not permit the prediction of relapses of the disease or of its outcome. The method does not suggest the use of antiviral immunoglobulins for the discovery of cells infected by viruses, including immune system cells, in the dynamic of the development of cardiovascular illness, which does not allow the chance to predict the development of cardiovascular disease in healthy people long before the appearance of the first clinical indicators of atherosclerosis or evaluate the severity of the development of the illness in cardiovascular disease patients according to the level of viral pressure on the immune system.

### DISCLOSURE OF THE INVENTION

The invention's task was to develop a diagnostic method for the prediction of the development and control of the effectiveness of the treatment of cardiovascular illnesses that allows interactive control of the effectiveness of the treatment of atherosclerosis patients by standard methods in combination with antiviral substances, to predict the intensity and number of complications, and to predict the danger of the appearance of atherosclerosis and its complications in practically healthy people by the level of infected immunocytes.

The task set is addressed through a diagnostic method for the prediction of the development and control of the effectiveness of the treatment of cardiovascular, in which patient tissue samples are taken (immunocytes and erythrocytes from venous and capillary blood, smears and prints from atherosclerotic plaques after surgical intervention, and cells from urinary and salivary sediment), microdrugs are prepared, specific anti-viral immunoglobulins are processed (for types 1 and 2 herpes viruses, the cytomegalovirus, the herpes Zoster virus, the Epstein-Barr virus, the herpes 6 virus, or the combinations thereof), the number of cells infected by two or more viruses before the beginning of treatment, during treatment, and after treatment are determined with the application of anti-viral therapy, and the dynamic of the change in the number of infected cells and their interrelationships are established: if the number of cells infected by two or more viruses, among which cytomegalovirus must be found, exceeds 50±10% in patients with cardiovascular disease, a diagnostic conclusion of the threat of atherosclerosis complications such as myocardial infarction and stroke, as well as of complications such as arrhythmia, thrombic embolisms, severe left ventricular failure, repeated myocardial infarction, cardiogenic shock, and a high possibility of a fatal result; the method can also be used in patients with cardiovascular diseases after treatment through a method of a combination of traditional methods and the use of anti-viral drugs and a change is seen in the percentage of infected cells after treatment if the number of cells infected by two or more viruses decreases by more than 20±10% in repeat diagnostics, the treatment is considered successful, whereas if a decrease by 20±10% in the number of cells infected by cytomegalovirus and any other aforementioned virus in repeat diagnostics is not seen, a conclusion is drawn on the ineffectiveness of the therapy applied in that period and the need to change the antiviral therapy scheme.

### EXAMPLES OF INVENTION IMPLEMENTATION

### Example 1

Patient L., 52 years old, came to the clinic with a diagnosis of ischemic heart disease. Exertional angina, functional class III. Postinfarction cardiosclerosis (myocardial infarction in 1981). Atherosclerosis of the coronary arteries. Circulatory deficiency stage IIA. Cerebral atherosclerosis. Chronic cerebral impairment. Symptomatic hypertension. At arrival, complained of a squeezing pain in the area of the heart with radiation to the right hand, which arise both at rest and during physical activity (up to 4-5 times per day) and were relieved by taking nitroglycerin (up to 8 times per day), as well as shortness of breath while walking, vertigo, periodic headaches, ringing in the ears, irritability, and insomnia. IIF detected cytomegalovirus (60% of cells infected) and EBV (70% of cells infected) in the patient's immunocytes. The patient was prescribed treatment: anti-angina drugs (Nitrosorbidum 30 mg/day, Corinfar 30 mg/day; also has hypotensive activity), and Valacyclovir 2 tablets (1.0 g) three times a day. As a result of the treatment, a decrease in the blood serum of total cholesterol from 6.71 mmol/l to 3.26 mmol/l, of triglycerides from 3.4 mmol/l to 1.04 mmol/l, of b-lipoproteins from 770 conditional units to 310 conditional units, cholesterol/LDL from 3.99 mmol/l to 3.68 mmol/l, and LDL from 1.55 mmol/l to 0.47 mmol/l. The atherogenesis index fell from 4.74 to 3.38, and there was an increase in cholesterol/HDL from 1.17 mmol/l to 1.20 mmol/l. During the treatment process, a slowing in the free-radical acidization of lipids was seen; the content of serum malondialdehyde fell from 0.50 mcmol/ml to 0.31 mcmol/ml, diethynoid conjugates fell from 1.332 mcmol/ml to 0.70 mcmol/ml, triethynoid conjugates declined from 0.23 mcmol/ml to 0.14 mcmol/ml; the a-tocopherol content recovered from 2.69 mcmol/ml to 4.32 mcmol/ml; the glutathione reductase activity went from 12.4 mcmol/l h to 22.0 mcmol/l h. On the electrocardiogram, a decrease in the T index from 35.0 to 0, in the SST from 11.0 to 0, and in the NST from 10.0 to 0 were seen. Hemodynamic indicators also improved; the total peripheral vascular resistance decreased from 2917.9 dyn s cm⁻⁵ to 2673.6 dyn s cm⁻⁵; the specific peripheral resistance went from 74.53 conditional units to 65.97 conditional units; the energy loss fell from 15.96 conditional units to 10.37 conditional units; the flow rate increased from 126.5 ml/sec to 169.71 ml/sec, and the capacity of the left ventricle improved from 2.02 W to 2.29 W. IIF: in the blood immunocytes, CMV (20%) and EBV (50%) were found. Within seven weeks, the patient's condition had improved significantly. Angina pain and shortness of breath not present; nitroglycerine tablets not used. Vertigo and headaches had decreased.

### Example 2

Patient M., 60 years old. Diagnosis: ischemic heart disease: Postinfarction cardiosclerosis (1999). Stage 3 hypertonic disease. Stage-1 circulatory deficiency. Treatment: metoprolol (Corvitol) 50 mg 2×/day, enalapril (Renitec) 10 mg 2×/day, aspirin 80 mg/day, simvastatin (Zocor) 10 mg in the evening. After six months of simvastatin 10 mg/day, the following indicators of the lipid and liver complexes were obtained: total cholesterol 4.9 mmoles/1 (goal indicator 5.0 mmoles/l), triglycerides 1.55 mmoles/l, GOT 110 IU/l (norm 10-39 IU/l), GPT 100 IU/l (norm 10-35 IU/l).

IIF detected cytomegalovirus (50% of cells infected) and EBV (50% of cells infected) in the patient's immunocytes.

After Valacyclovir 2 tablets (1.0 g) 3×/day for 3 courses of 7 days, the following indicators were obtained: total cholesterol 4.7 mmoles/1 (goal value 5.0 mmoles/l), triglycerides 1.3 mmoles/l, GOT 22 IU/l (norm 10-39 IU/l), GPT 32 IU/l (norm 10-35 IU/l). IIF: in the blood immunocytes, CMV was not present; EBV 10%.

### Example 3

Patient Ch., 40 years old. Diagnosis: ischemic heart disease: Postinfarction cardiosclerosis (1998). Stage 1 circulatory deficiency. Treatment: metoprolol (Corvitol) 25 mg 2×/day, aspirin 80 mg/day, simvastatin (Zocor) 10 mg in the evening. After five months of simvastatin 10 mg/day, the following indicators of the lipid and liver complexes were obtained: total cholesterol 5.6 mmoles/1 (goal indicator 5.0 mmoles/l), triglycerides 2.55 mmoles/l, GOT 89 IU/1 (norm 10-39 IU/l), GPT 96 IU/l (norm 10-35 IU/l).

After the treatment, IIF detected cytomegalovirus (50% of cells infected) and HSV-1 (70% of cells infected) in the patient's immunocytes.

After Valacyclovir 1 g 3×/day for 5 courses of 7 days each with 7-day intervals, the following indicators of the lipid and liver complexes were obtained: total cholesterol 4.9 mmoles/l, triglycerides 1.95 mmoles/l, GOT 38 IU/l (norm 10-39 IU/l), GPT 30 IU/l (norm 10-35 IU/l).

After the treatment, IIF detected cytomegalovirus (10% of cells infected) and HSV-1 (30% of cells infected) in the patient's immunocytes.

As the examples provided indicate, the combination of a standard treatment scheme with Valacyclovir after correctly conducted virological diagnostics according to the method being patented with an increase in the level of transaminase while statins are being taken allows an increase in the effectiveness of atherosclerosis and dyslipidemia treatment. Thus in the process of treatment, in the patient who took Valacyclovir, lipidogram, peroxide acidization of lipids, electrocardiography, and tetrapolar rheography indicators normalized.

### Example 4

Patient L., 48 years old. Diagnosis: ischemic heart disease: Postinfarction cardiosclerosis (2006). Stage 3 hypertonic disease. Stage 1 circulatory deficiency. Treatment: Corvitol 50 mg 2×/day, Renitec 10 mg 2×/day, aspirin 80 mg/day, Zocor 20 mg in the evening. After six months of Zocor 20 mg/day, the following indicators of the lipid and liver complexes were obtained: total cholesterol 3.9 mmoles/1 (goal indicator 4.0 mmoles/l), triglycerides 1.4 mmoles/l, GOT 67 IU/l (norm 10-39 IU/l), GPT 88 IU/l (norm 10-35 IU/l). As a result of IIF study of the blood immunocytes, antigens to the Epstein-Barr virus (50% of cells infected), cytomegalovirus (30% of cells infected), and herpes type 1 virus (60% of cells infected) were found.

The patient then underwent combination therapy with anti-viral drugs: immunoglobulin to treat the EBV at a dosage of 13 ml of a 10% solution immediately (7 ml per injection) intramuscularly once a week three times; Valacyclovir to treat the CMV at a dosage of 2 tablets (1.0 g) 3 times a day in three courses of seven days each; Laferobion 3 million IU once per day for seven days in a row. The following indicators were obtained: total cholesterol 1.5 mmoles/1, triglycerides 1.3 mmoles/1, GOT 20 IU/l (norm 10-39 WA), GPT 35 IU/l (norm 10-35 IU/l). Repeat IIF study after two months did not discover antigens to any of the viruses discovered earlier in the immunocytes.

### INDUSTRIAL APPLICABILITY OF THE METHOD

This method of diagnosis allows the precise differentiation of attacks on the immune system leading to the persistence of cytomegalovirus and other herpes viruses in the vascular wall with resultant development of atherosclerosis, which permits the timely liquidation of this condition and the prevention of the appearance and development of atherosclerosis and its consequences, such as severe myocardial infarctions and strokes, long before they arise. It also allows the prediction of the risk of the further development of the pathology and the need for the combined use of anti-viral drugs in atherosclerosis patients. All of the test systems can be produced by the pharmaceutical industry; in order to implement the method in a clinic, it is sufficient to enter the method in the diagnostic standards for the pathology of the cardiovascular system.

### REFERENCES

1 Braunwald E. Cardiovascular Medicine at the Turn of the Millennium: Triumphs, Concerns, and Opportunities//N. Engl. J. Med.—1997.—Vol. 337, N 19.—P. 1360-1369.
2 Highlights on Health in Ukraine//WHO Regional Office for Europe.—2005.—http://www.euro.who.int/document/e88285.pdf
3 Goldschmidt-Clermont P. J., Creager M. A., Lorsordo D. W. et al. Atherosclerosis 2005: Recent Discoveries and Novel Hypotheses//Circulation.—2005.—Vol. 112, N 21.—P. 3348-3353.
4 Lusis A. J. Atherosclerosis//Nature.—2000.—Vol. 407, N 6801.—P. 233-241.
5 Ross R. Atherosclerosis—An Inflammatory Disease//N. Engl. J. Med.—1999.—Vol. 340, N 2.—P. 115-126.
6 Lusis A. J. Atherosclerosis//Nature.—2000.—Vol. 407, N 6801.—P. 233-241.
7 Rauscher F. M., Goldschmidt-Clermont P. J., Davis B. H et al. Aging, Progenitor Cell Exhaustion, and Atherosclerosis//Circulation.—2003.—Vol. 108, N 4.—P. 457-463.
8 Serrano A. L., Andres V. Telomeres and Cardiovascular Disease: Does Size Matter?//Circ. Res—2004.—Vol. 94, N 5.—P. 575-584.
9 Goldschmidt-Clermont P. J., Creager M. A., Lorsordo D. W. et al. Atherosclerosis 2005: Recent Discoveries and Novel Hypotheses//Circulation.—2005.—Vol. 112, N 21.—P. 3348-3353.
10 Hansson G. K. Inflammation, Atherosclerosis, and Coronary Artery Disease//N. Engl. J. Med.—2005.—Vol. 352, N 16.—P. 1685-1695.
11 http://en.wikipedia.org/wild/Atherosclerosis
12 Epstein S. E., Zhou Y. F., Zhu J. Infection and Atherosclerosis: Emerging Mechanistic Paradigms//Circulation.—1999.—Vol. 100, N 4.—P. 20-28.
13 Stacey L. K., Lemon S. M., Najafi M., Editors The Infectious Etiology of Chronic Diseases: Defining the Relationship, Enhancing the Research, and Mitigating the Effects—Washington, D.C.: The National Academic Press, 2004.—215 p.
14 Virchow R. Der atheromatose prozess der arterien//Wien. Med. Wochenschr.—1856.—Vol. 6.—P. 809-812, 825-827
15 Fabricant C. G., Fabricant J., Litrenta M. M., Minick C. R. Virus-induced atherosclerosis//J. Exp. Med.—1978.—Vol. 148, N1.—P. 335-340.
16 Gandzha I M. Viral infection, atherosclerosis, and ischemic heart disease//Lik. sprava. Vracheb. delo.—2001.—No 1.—pp. 65-67.
17 Bruggeman C. A. Does Cytomegalovirus Play a Role in Atherosclerosis?//Herpes.—2000.—Vol. 7, N 2.—P. 51-54.
18 Grahame-Clarke C. Human cytomegalovirus, endothelial function and atherosclerosis//Herpes.—2005.—Vol. 12, N 2.—P. 42-45.
19 Grahame-Clarke C, Chan N. N, Andrew D. et al. Human cytomegalovirus seropositivity is associated with impaired vascular function//Circulation.—2003.—Vol. 108, N 6.—P. 678-683.
20 Reinhardt B., Minisini R., Mertens T Opinion article: cytomegalovirus is a risk factor in atherogenesis//Herpes.—2002.—Vol. 9, N1.—P. 21-23.
21 Schlitt A., Blankenberg S., Weise K. et al. Herpesvirus DNA (Epstein-Barr virus, herpes simplex virus, cytomegalovirus) in circulating monocytes of patients with coronary artery disease//Acta Cardiol.—2005.—Vol. 60, N 6.—P. 605-610.
22 Sorlie P. D., Adam E., Melnick S. L. et al. Cytomegalovirus/herpesvirus and carotid atherosclerosis: the ARIC Study//J. Med. Virol.—1994.—Vol. 42, N1.—P. 33-37.
23 Kotronias D. F., Kapranos N. Herpes simplex virus as a determinant risk factor for coronary artery atherosclerosis and myocardial infarction//In Vivo.—2005.—Vol, 19, N 2.—P. 351-357.
24 Visser M. R., Vercellotti G. M. Herpes simplex vims and atherosclerosis//Eur. Heart J.—1993.—Vol. 14 Suppl K.—P. 39-42.
25 Morre S. A., Stooker W., Lagrand W. K et al. Microorganisms in the aetiology of atherosclerosis//J. Clin. Pathol.—2000.—Vol. 53, N 9.—P. 647-654.
26 Schlitt A., Blankenberg S., Weise K. et al. Herpesvirus DNA (Epstein-Barr virus, herpes simplex virus, cytomegalovirus) in circulating monocytes of patients with coronary artery disease//Acta Cardiol.—2005.—Vol. 60, N 6.—P. 605-610.
27 Dunne M. Infectious Agents and Cardiovascular Disease//The Infectious Etiology of Chronic Diseases: Defining the Relationship, Enhancing the Research, and Mitigating the Effects—Washington, D.C: The National Academic Press, 2004.—215 p.
28 Reinhardt B., Minisini R., Mertens T Opinion article: cytomegalovirus is a risk factor in atherogenesis//Herpes.—2002.—Vol. 9, N1.—P. 21-23.
29 Vatutin N T, Chupina V A. Infection as a factor in the development of atherosclerosis and its complications//Cardiology.—2000.—No 2.—pp. 67-71.
30 Lindholt J. S., Stovring J., Andersen P. L. et al. A review of macrolide treatment of atherosclerosis and abdominal aortic aneurysms//Curr. Drag. Targets Infect. Disord.—2003.—Vol. 3, N1.—P. 55-63.
31 Pechere J. C. New perspectives on macrolide antibiotics//Int. J. Antimicrob. Agents.—2001.—Vol. 18, Suppl 1.—P. S93-97.
32 Anderson J. L., Muhlestein J. B. Antibiotic trials for coronary heart disease//Tex. Heart. Inst. J.—2004.—Vol. 31, N1.—P. 33-38.
33 Andraws R., Berger J. S., Brown D. L. Effects of antibiotic therapy on outcomes of patients with coronary artery disease: a meta-analysis of randomized controlled trials//JAMA.—2005.—Vol. 293, N 21.—P. 2641-2647.
34 Kuppuswamy V. C, Gupta S. Antibiotic therapy for coronary heart disease: the myth and the reality//Timely Top. Med. Cardiovasc. Dis.—2006.—Vol. 10.—P. E2.
35 Muhlestein J. B. Antibiotic treatment of atherosclerosis//Curr. Opin. Lipidol.—2003.—Vol. 14, N 6.—P. 605-614.
36 Goldschmidt-Clermont P. J., Creager M. A., Lorsordo D. W. et al. Atherosclerosis 2005: Recent Discoveries and Novel Hypotheses//Circulation.—2005.—Vol. 112, N 21.—P. 3348-3353
37 Meng C. Q., Somers P. K, Rachita C. L. et al. Novel phenolic antioxidants as multifunctional inhibitors of inducible VCAM-1 expression for use in atherosclerosis//Bioorg. Med. Chem. Lett.—2002.—Vol. 12, N 18.—P. 2545-2548.
38 Cull V. S., Bartlett E. J., James C M. Type I interferon gene therapy protects against cytomegalovirus-induced myocarditis//Immunology.—2002.—Vol. 106, N 3.—P. 428-437.
39 Patent application publication No. US2007/0099239. Methods and compositions for diagnosis and monitoring of atherosclerotic cardiovascular disease/Raymond Tabibiazar, Philip Tsao, Thomas Quertermous et al.

1. A diagnostic method for the prediction of the development of cardiovascular diseases, in which samples of patients' tissues are taken, microdrugs are prepared, specific immunoglobulins are processed using an immunofluorescence method, and the percentage of fluorescing cells are determined and counted, distinguished by the fact that in the capacity of specific immunoglobulins, antiviral immunoglobulins are used and the quantity of cells infected by two or more viruses is determined.

2. A diagnostic method for the prediction of the development of cardiovascular diseases according to claim 1, in which the quantity of cells infected by any two or more viruses, among which cytomegalovirus must be found, exceeds 50±10% in patients without signs of cardiovascular disease and a diagnostic conclusion has been reached on a high level of danger of atherosclerosis.

3. A diagnostic method for the prediction of the development of cardiovascular diseases according to claim 1, in which the quantity of cells infected by any two or more viruses, among which cytomegalovirus must be found, exceeds 50±10% in patients with cardiovascular diseases, and a diagnostic conclusion has been reached on a high level of danger of atherosclerosis complications such as myocardial infarction or stroke, as well as complications such as arhythmia, thrombic embolism, severe left ventricular failure, repeat myocardial infarction, cardiogenic shock, and a high likelihood of a fatal outcome.

4. A diagnostic method for the prediction of the development of cardiovascular diseases according to any one of claims 1-3, in which immunocytes from venous blood are used as patient tissue samples.

5. A diagnostic method for the prediction of the development of cardiovascular diseases according to any one of claims 1-3, in which immunocytes from capillary blood are used as patient tissue samples.

6. A diagnostic method for the prediction of the development of cardiovascular diseases according to any one of claims 1-3, in which erythrocytes from venous blood are used as patient tissue samples.

7. A diagnostic method for the prediction of the development of cardiovascular diseases according to any one of claims 1-3, in which erythrocytes from capillary blood are used as patient tissue samples.

8. A diagnostic method for the prediction of the development of cardiovascular diseases according to any one of claims 1-3, in which in the capacity of patient tissue samples, samples of the material from the atherosclerotic plaque taken after surgical intervention are used.

9. A diagnostic method for the prediction of the development of cardiovascular diseases according to any one of claims 1-3, in which in the capacity of patient tissue samples, samples of the material from the regions neighboring atherosclerotic plaque area taken after surgical intervention are used.

10. A diagnostic method for the prediction of the development of cardiovascular diseases according to any one of claims 1-3, in which cells from patient urocheras are used as patient tissue samples.

11. A diagnostic method for the prediction of the development of cardiovascular diseases according to any one of claims 1-3, in which cells from patient salivary sediment are used as patient tissue samples.

12. A diagnostic method for the prediction of the development of cardiovascular diseases according to claim 1, in which various combinations of samples in claims 4-11 are used as patient tissue samples.

13. A diagnostic method for the prediction of the development of cardiovascular diseases according to claim 12, in which immunoglobulins against the type 1 human herpes virus are used as specific immunoglobulins.

14. A diagnostic method for the prediction of the development of cardiovascular diseases according to claim 12, in which immunoglobulins against the type 2 human herpes virus are used as specific immunoglobulins.

15. A diagnostic method for the prediction of the development of cardiovascular diseases according to claim 12, in which immunoglobulins against the herpes Zoster virus are used as specific immunoglobulins.

16. A diagnostic method for the prediction of the development of cardiovascular diseases according to claim 12, in which immunoglobulins against human cytomegalovirus are used as specific immunoglobulins.

17. A diagnostic method for the prediction of the development of cardiovascular diseases according to claim 12, in which immunoglobulins against the Epstein-Barr virus are used as specific immunoglobulins.

18. A diagnostic method for the prediction of the development of cardiovascular diseases according to claim 12, in which immunoglobulins against the type 6 human herpes virus are used as specific immunoglobulins.

19. A diagnostic method for the prediction of the development of cardiovascular diseases according to claim 12, in which various combinations of immunoglobulins according to any one of claims 13-18 are used as specific immunoglobulins.

20. A diagnostic method for the control of the effectiveness of the treatment of cardiovascular diseases, in which patient tissue samples are taken, microdrugs are prepared, specific antiviral immunoglobulins are processed using the immunofluorescence method, and the percentage of fluorescing cells is determined, distinguished by the fact that antiviral immunoglobulins are used as specific immunoglobulins, the number of cells infected by two or more viruses before the beginning of treatment, in the process of treatment, and after treatment with the application of antiviral therapy are determined, and the dynamic of the change in the number of infected cells and their interrelationships are established: when the number of cells infected by cytomegalovirus and any other virus decreases by more than 20±10%, the treatment is considered successful, whereas an absence of changes or an increase in the number of cells infected by cytomegalovirus and any other viruses is considered an indication of unsuccessful treatment.

21. A diagnostic method for the control of the effectiveness of the treatment of cardiovascular diseases according to claim 20 that is applied to patients with cardiovascular diseases after their treatment with a combination of traditional methods and the use of anti-viral drugs and determines the change in the percentage of infected cells after treatment.

22. A diagnostic method for the control of the effectiveness of the treatment of cardiovascular diseases according to claim 20, in which a decrease in the number of infected cells of 20±10% or more is seen in a repeat diagnosis for two or more viruses, among which one must be cytomegalovirus, and a conclusion is reached on the effectiveness of the therapy conducted.

23. A diagnostic method for the control of the effectiveness of the treatment of cardiovascular diseases according to claim 20, in which a decrease in the number of infected cells of 20±10% or more is not seen in a repeat diagnosis for cytomegalovirus and any other virus listed earlier, and a conclusion is reached on the ineffectiveness of the therapy conducted in that period and the necessity of changing the antiviral therapy scheme.

24. A diagnostic method for the prediction of the development of cardiovascular diseases according to any one of claims 20-23, in which immunocytes from venous blood are used as patient tissue samples.

25. A diagnostic method for the prediction of the development of cardiovascular diseases according to any one of claims 20-23, in which immunocytes from capillary blood are used as patient tissue samples.

26. A diagnostic method for the prediction of the development of cardiovascular diseases according to any one of claims 20-23, in which erythrocytes from venous blood are used as patient tissue samples.

27. A diagnostic method for the prediction of the development of cardiovascular diseases according to any one of claims 20-23, in which erythrocytes from capillary blood are used as patient tissue samples.

28. A diagnostic method for the prediction of the development of cardiovascular diseases according to any one of claims 20-23, in which in the capacity of patient tissue samples, prints of the material from the atherosclerotic plaque taken after surgical intervention are used.

29. A diagnostic method for the prediction of the development of cardiovascular diseases according to any one of claims 20-23, in which in the capacity of patient tissue samples, prints of the material from the regions neighboring the atherosclerotic plaque area taken after surgical intervention are used.

30. A diagnostic method for the prediction of the development of cardiovascular diseases according to any one of claims 20-23, in which patient urinary sediment cells are used as patient tissue samples.

31. A diagnostic method for the prediction of the development of cardiovascular diseases according to any one of claims 20-23, in which patient salivary sediment cells are used as patient tissue samples.

32. A diagnostic method for the prediction of the development of cardiovascular diseases according to any one of claims 20-23, in which samples according to any one of claims 24-31 in various combinations are used as patient tissue samples.

33. A diagnostic method for the prediction of the development of cardiovascular diseases according to claim 32, in which immunoglobulins against the type 1 human herpes virus are used as specific immunoglobulins.

34. A diagnostic method for the prediction of the development of cardiovascular diseases according to claim 32, in which immunoglobulins against the type 2 human herpes virus are used as specific immunoglobulins.

35. A diagnostic method for the prediction of the development of cardiovascular diseases according to claim 32, in which immunoglobulins against the herpes Zoster virus are used as specific immunoglobulins.

36. A diagnostic method for the prediction of the development of cardiovascular diseases according to claim 32, in which immunoglobulins against the human cytomegalovirus are used as specific immunoglobulins.

37. A diagnostic method for the prediction of the development of cardiovascular diseases according to claim 32, in which immunoglobulins against the Epstein-Barr virus are used as specific immunoglobulins.

38. A diagnostic method for the prediction of the development of cardiovascular diseases according to claim 32, in which immunoglobulins against the type 6 human herpes virus are used as specific immunoglobulins.

39. A diagnostic method for the prediction of the development of cardiovascular diseases according to claim 32, in which various combinations of immunoglobulins according to any one of claims 33-38 are used as specific immunoglobulins.
